(19) United States
(12) Patent Application Publication
(10) Pub. No.: US 2004/0231676 A1
(43) Pub. Date: Nov. 25, 2004

(54) Prophylactic article useful for both protection and diagnosis and method for use and production thereof

(76) Inventors: Alexander Schoenfeld, Herzeliya Pituach (IL); Emil Katz, Savion (IL)

Correspondence Address: LOWE HAUPTMAN GILMAN AND BERNER, LLP, 1700 DIAGONAL ROAD, SUITE 300 /310, ALEXANDRIA, VA, 22314, US

(21) Appl. No.: 10/481,039

(22) PCT Filed: Jun. 12, 2002
(86) PCT No.: PCT/IL02/00455

(30) Foreign Application Priority Data
Jun. 18, 2001 (IL) 143810

(51) Int. Cl. A61F 6/02; A61F 6/04

(52) U.S. Cl. 128/842; 128/844

(57) ABSTRACT

The invention relates to a prophylactic article, useful for both protection and diagnosis of biological, chemical or physical specific conditions in mammals body comprising at least one probe, having means for diagnosis of said conditions. The invention also relates to a method for the use of prophylactic articles.

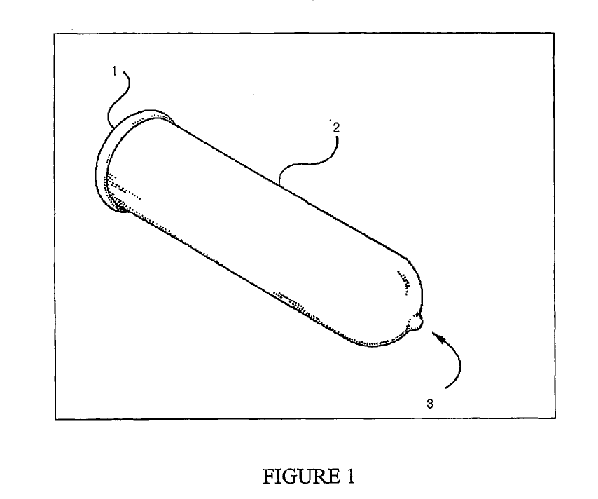

### FIELD OF THE INVENTION

The invention relates to prophylactic article comprising a probe useful for both protection and diagnosis of biological, chemical or physical specific conditions. More specifically, the invention relates to a condom useful for protection from sexually transmitted diseases (STDs), unintended pregnancy and of male sperm. The invention also relates to a verity of prophylactic articles, comprising of medical and surgical gloves, non-disposable gloves, wound dressings, artificial skin, implements, cloths and coatings and the method for their use and production.

### BACKGROUND OF THE INVENTION

Epidemiological diseases and their vectors, hazardous chemicals, and non-regular physical conditions might depress human health, and should be avoided by the presence of an effective barrier between the one person and the others, and between the person and the environment.

Humans might transmit diseases during their mutual sexual activities. It is hence estimated that 12 million people in the U.S. acquire some form of sexually transmitted diseases (STDs) each year. Overall, about one in four Americans has an STD. This category includes such conditions as herpes, syphilis, gonorrhea, trichomoniasis, chlamydia and HIV (AIDS), among others. STDs are most commonly spread when sexual partners engage in unprotected sex, that is, the infected genitalia (and surrounding area) of one partner comes into contact with the genitalia (and surrounding area) of another partner, thus transferring the infection. Although most STDs respond to some form of treatment, certain sexually transmitted pathogens cause cervical, liver, and other cancers, while infections in pregnant women can cause spontaneous abortion, stillbirth, pre-term delivery, and illness among infants.

An example of a commonly used and highly effective barrier method is the use of condom by the male partner or of a similar barrier used by the female partner to provide complete isolation against transfer of bodily fluids from one partner to the other. Such devices are effective for prevention of transmission of STDs and for contraception, provided that the physical barrier remains intact. In some cases however, the condom or the like may develop microscopic leaks or may contain small and perhaps unnoticeable perforations, which can permit the transfer of viruses and microorganisms or of sperm across the barrier. In other cases the condom may rupture and permit substantial transfer of bodily fluids with the consequence of possible infection or conception. Although gross rupture of a condom is relatively rare, it has been found that leaks sufficient to permit the escape of microorganisms in infectious amounts are by no means uncommon. Accordingly, the recent emergence of more serious and even fatal STDs such as AIDS has caused concern that even relatively reliable total barrier methods may not be satisfactory.

The condom was hence proven effective providing prophylactic and birth control. The male condom is the most popular prophylactic article and comprises a tubular shaft portion that is roughly cylindrical and that covers the shaft of the penis and a scrotal portion that covers the scrotum. The female condom is less common. Thus, n this invention, both male condom and female condoms, made of latex, polyurethane, rubber or other polymer or elastomer will denoted as ‘condom’, and only for the purpose of simplicity the male condom will be exclusively described. The condom is fashioned into a non-rigid, pliant construction. The male condom is closed at one end and includes an optional reservoir tip for containing semen, and is open at the other end for admitting the penis and the scrotum. The periphery of the open end is preferably finished with a lip or band. The scrotal portion includes a scrotal sac or pouch that extends downward from the flexible open end, so that once the penis is effectively fitted into the tubular shaft portion, the scrotum easily and comfortably enters the scrotal sac.

Condoms are widely used in various shapes and polymeric compositions for the protection from STDs and form unintended pregnancy. Some patents have been issued condoms of dual action: obtaining (i) an effective barrier, and (ii) an active antimicrobial materials (U.S. Pat. No. 5,878,747; U.S. Pat. No. 6,183,764) and intravaginally placed spermatocides that have been used for contraception, alone or in combination with barrier methods (U.S. Pat. No. 6,074,671).

Condoms are effective for prevention of transmission of STDs and for contraception, provided that the physical barrier remains intact. In some cases however, the condom or the like may develop microscopic leaks or may contain small and perhaps unnoticeable perforations, which can permit the transfer of viruses and microorganisms or of sperm across the barrier. In other cases the condom may rupture and permit substantial transfer of bodily fluids with the consequence of possible infection or conception. Although gross rupture of a condom is relatively rare, it has been found that leaks sufficient to permit the escape of microorganisms in infectious amounts are by no means uncommon. Accordingly, the recent emergence of more serious and even fatal STDs has caused concern that even relatively reliable total barrier methods may not be satisfactory.

The methods of disease detection are divided in the present invention into two general types: diagnosis and screening. Diagnosis will be defined as the method whereby a physician determines the nature of a disease based upon the patient's signs and symptoms. Screening will be defined as the method of suggesting the presence, or the absence, of a particular disease, or class of diseases, in a patient. When a screening test indicates that a patient does not have a disease, in many cases the need for further diagnostic testing has been eliminated.

In addition, screening provides a way for patients to avoid the cost, discomfort or undesired publicity associated with the more invasive procedures often necessary to collect the samples required for diagnostic testing. Nevertheless, a multi-purpose prophylactic article, useful for both protection and diagnosis of biological, chemical or physical specific conditions was never been presented.

### SUMMARY OF THE INVENTION

It is thus the object of the present invention to provide an improved prophylactic article, useful for both protection and diagnosis of biological, chemical or physical specific conditions in mammals body comprising at least one probe, having means for diagnosis of said conditions

It is also in the scope of the present invention to provide an improved prophylactic article, as defined above, wherein the prophylactic article is a male condom or a female condom, useful for protection from sexually transmitted diseases (STDs) and from unintended pregnancy; comprising at least one probe that enables rapid and sensitive diagnosis of said STDs.

It is also in the scope of the present invention to provide an improved prophylactic article, comprising condom as defined above, wherein said probe comprising at least one biological substance of the group of immune complexes, proteins, antibodies, antigens and nucleotides; and means for detecting whether the specific binding substance is bound to at least one of the group: an STD, STD's expression and to an STD vector, microorganism particle or antigen.

It is also in the scope of the present invention to provide an improved prophylactic article, comprising condom as defined above, whereas said probe have means of diagnosis of at least one of the known STD comprising by not limited to Acquired Immune Deficiency Syndrome (i.e., AIDS); Bacterial Vaginosis (e.g., Gardnerella vaginitis); chlamydia; Pediculosis pubis (e.g., crab lice); Neisseria gonorrhoeae; Hepatitis B; herpes virus; Human Papillomavirus (i.e., HPV); Molluscum Contagiosum; Scabies; Syphilis; Trichomoniasis (e.g., Trich).

It is also in the scope of the present invention to provide an improved prophylactic article, comprising condom as defined above, whereas said probe having means for sensitive screening for the presence of male sperm at the vaginal side of said article, and wherein said article is comprising a specific binding substance and means for detecting whether the specific binding molecule is bound to male sperm. It is still an object of the present invention to provide an improved prophylactic article, condom as defined above, whereas said probe is a sperm-specific antigen.

It is also in the scope of the present invention is to provide an improved prophylactic article, as defined above, wherein said article is selected from the group of surgical gloves, non-disposable gloves, wound dressings, artificial skin, implements, cloths and coatings.

It is also in the scope of the present invention to provide an improved prophylactic article, comprising surgical glove as defined above, whereas said probe have means for protection and for the diagnosing of various conditions related to the presence of at least one from the group of contaminated diseases, disease vectors, hazardous chemicals, and physical conditions.

It is still another object of the present invention to provide an improved method for the use prophylactic articles as defined above, useful for both protection and for biological, chemical or physical specific conditions as described in all claims above, wherein comprising (i) locating an assay probe on said prophylactic article for the detection of condition to be tested in one or more sites on said article and fitting by means of adjustment said article on an object organ, wherein said object organ is defined as the target organ said prophylactic article is to be administrate and adjusted, comprising by not limited to male or female sex organs, hands, head, and body skin; (ii) bringing at least one surface of said organ to be tested and at least one surface of said article into contact for an efficient period of time; (iii) measuring biological, chemical or physical specific conditions diagnosing by means of probe detecting, screening, or analyzing of said condition.

It is also in the scope of the present invention to provide an improved method for the use said prophylactic article, as defined above, comprising male condom or female condom, useful for both protection and for diagnosing at least one conditions of STDs and presence of male sperm in the vaginal side of said article; wherein comprising (i) locating an assay probe on said condom for the detection of condition to be tested in one or more sites on said article and fitting by means of adjustment said article on an object organ; (ii) bringing at least one surface of said organ to be tested and at least one surface of said article into contact for an efficient period of time; (iii) measuring said condition by means of probe detecting, screening, or analyzing of said condition.

It is still in the scope of the present invention to provide an improved method for the use prophylactic article, comprising surgical gloves, working gloves and non-disposable gloves as defined above, useful for protection and for the diagnosing of various conditions related to the presence of at least one from the group of contaminated diseases, disease vectors, hazardous chemicals, and physical conditions, wherein comprising (i) locating an assay probe on said article for the detection of condition to be tested in one or more sites on said article and fitting by means of adjustment said article on an object organ; (ii) bringing at least one surface of said organ to be tested and at least one surface of said article into contact for an efficient period of time; (iii) measuring said condition by means of probe detecting, screening, or analyzing of said condition.

Finally, it is also in the scope of the present invention to provide an improved method for the production of prophylactic article as defined in any of the claims above, comprising (i) preparing the prophylactic article by an organic polymer selected from the group consisting of polyvinyl chloride, latex, polyurethane, polyacrylate, polyester, polyethylene terephthalate, polymethacrylate, silicone rubber, silicon elastomers, polystyrene, polycarbonate and polysulfones; (ii) absorbing, immobilizing, entrapping or inserting at least one probe onto the organic polymer at the proper location; and optionally (iii), incorporating suitable diagnosis reference onto the organic polymer at predetermined location.

### DETAILED DESCRIPTION OF THE PREFERRED EMBODIMENTS OF THE INVENTION

In accordance with the present invention, a novel probe comprising prophylactic article has been developed and proved effective for both protection and diagnosis of biological, chemical or physical specific conditions.

In one preferred embodiment of the present invention is a prophylactic article, comprising mammalian male or female polymeric condom (hereby denoted “condom”), useful for protection from various STDs, as is defined below, and unintended pregnancy; comprising at least one probe that enables rapid and sensitive diagnosis of said STDs.

Said condom is highly effective for humans, yet is also efficient for other mammals, especially as a collecting sperm article comprising a probe for veterinary diseases and sperm condition for mating horses, bulls, goats and dogs.

In the present invention an STD will defined as at least one known sexual transmitted disease, comprising by not limited to Acquired Immune Deficiency Syndrome (i.e., AIDS); Bacterial Vaginosis (e.g., Gardnerella vaginitis); chlamydia; Pediculosis pubis (e.g., crab lice); Neisseria gonorrhoeae; Hepatitis B; herpes virus; Human Papillomavirus (i.e., HPV); Molluscum Contagiosum; Scabies; Syphilis, and Trichomoniasis (e.g., Trich).

In another preferred embodiment of the present invention is a condom as described above, whereas a probe enables rapid and sensitive diagnosis of said STDs. This probe comprises a specific binding molecule and means for detecting whether the specific binding molecule is bound to an STD or to an STD vector, such as certain viruses, bacteria, fungi and other pathogenic organisms.

One preferred embodiment of the present invention is a condom hereto described, comprising means for partners having mutual sexual intercourse to screen whether at least one of said partners is a vector of said STDs. Said screening may be with or without all partners awareness and permission. Still preferred embodiment is the screening for AIDS vectors among sexual partners.

Means for detecting said STDs are various and comprise, yet not limited, to covalent binding, entrapment, electrostatic connection and adsorption can be used for immobilizing antigen while retaining its activity. Advantageously, when the entrapment method is employed, little lowering of the activity is observed. It is thus another preferred embodiment of the present invention to incorporate suitable probe onto organic polymers by means of immobilization, entrapment and binding by various methods available in prior art.

In the present invention the term ‘probe incorporation’ comprises, yet not limited to incorporation of the probe as defined above in one or more layers, in one or more interfaces of said article, in the inner, outer or both sides, in a particular site, such as the tip of a male condom or the finger edge of a surgical glove, or spread by means of immersing or doping said probe in various of sites on, in or near said article.

The detection and screening are preferably provided by means of binding a probe with at least one characteristic of the condition been tested, comprising biological substances, as specific immune complexes, proteins (and for example lipoproteins, glycoproteins, amino-acids, peptides and synthesized active sites of said proteins etc.), antibodies and monoclonal antibodies, antigens, oligonucleotides, polynucleotides etc., chemical species and phenomena, comprising specific elements, molecules, and reactions; and physical parameters, comprising specific parameters, such as heat, presence of radioactivity, X-ray, MRI and ultrasonic activities.

Said probe is indicative for said tested condition preferably by labeling the state of said condition with light transparency, fluorescent labels, chemiluminescent labels, or chromophore labels. Nevertheless, other labels are with no means to be excluded, such as changes of conductivity, and preferably alternation of conductivity parameters related with the polymeric article; agglomerates formation; electric signals transmission and electromagnetic data acquisition etc.

Another preferred embodiment of the present invention is a prophylactic article, comprising male condom or female condom as described above, whereas said probe enables rapid and sensitive diagnosis the presence male sperm at the vaginal side of said article. Said condom preferably comprises a specific probe and means for detecting whether a specific binding molecule of the probe is bound to male sperm. Thus, wherein an undesirable leakage of male sperm via said condom is occur, it is detected by said probe, and the use of efficient birth controlling means may be considered.

Another preferred embodiment of the present invention is a prophylactic article, comprising, but not limited to surgical gloves, non-disposable gloves, useful for protection and for the diagnosing of various conditions related to the presence of at least one from the group of contaminated diseases, disease vectors, hazardous chemicals, and non-regular physical conditions. More specifically, one preferred embodiment of the present invention is an effective screening or diagnosing barrier, allowing an operator to work, and at the same time to be aware to presence of potential hazardous substances (e.g., heavy metals, toxic materials etc.), and extreme physical condition (e.g., heat, radioactivity, etc).

In another preferred embodiment of the present invention is a method for the use said prophylactic articles, useful for both protection and for biological, chemical or physical specific conditions as described in all claim above, wherein comprising (i) locating an assay probe on said prophylactic article for the detection of condition to be tested in one or more sites on said article, by means of probe immobilization, entrapment, insertion, binding etc,. (ii) fitting said barrier by means of adjustment the article on an object organ, and (iii) bringing at least one surface of said organ and at least one surface of said article into contact for an efficient period of time, and (iv) measuring biological, chemical or physical specific conditions diagnosing by means of probe detecting, screening, or analyzing of said condition. A method for the use said prophylactic article, comprising male condom or female condom, useful for both protection and for diagnosing the condition of STDs.

In another preferred embodiment of the present invention is method a method for the use aforementioned prophylactic article, comprising mammalian male or female condom, useful for both protection and for diagnosing the condition male sperm presence in the vaginal side of said condom.

In another preferred embodiment of the present invention is method a method for the use said prophylactic article, comprising, but not limited to surgical gloves, and non-disposable gloves useful for protection and for the diagnosing of various conditions related to the presence of at least one from the group of contaminated diseases, disease vectors, hazardous chemicals, and non-regular physical conditions.

It is still another preferred embodiment of the present invention is method a method for preparation of said prophylactic article, useful for both protection and diagnosis of biological, chemical or physical specific conditions, comprising (i) constituting said prophylactic article by an organic polymer selected from the group consisting of polyvinyl chloride, latex, polyurethane, polyacrylate, polyester, polyethylene terephthalate, polymethacrylate, silicone rubber, silicon elastomers, polystyrene, polycarbonate and polysulfones; (ii) absorbing, immobilizing, entrapping, binding or inserting at least one probe and onto the organic polymer at the proper location, and (iii), Due to color reaction, it is sometimes required to the article add such a reference providing a correct reference to the color changes of the probe. It is thus required in some of the probe system, to incorporate a suitable diagnosis reference onto the organic polymer at a proper location.

### EXAMPLES

### Example 1

AIDS Detecting Condom

AIDS (Acquired Immune Deficiency Syndrome) may be described more accurately as a “Spectrum of HIV Infection.” HIV (Human Immunodeficiency Virus) suppresses the immune system, which can lead to life-threatening opportunistic diseases. A person may be infected for years without any outward symptoms.

The detection of human immunodeficiency virus type 2 (denoted hereby as HIV-2) will be hereby presented as an example for AIDS screening method, and thus the present invention is by no means limited to this specific virus type. According to U.S. Pat. No. 6,197,496, which entire disclosure of this application is incorporated herein by reference, HIV-2 is utilizing multimeric forms of the envelope proteins gp300, p200, and p90/80. A purified immune complex comprising a protein and an antibody that binds with said protein, wherein the protein is selected from the group consisting of gp300 of HV-2_{ROD} having an apparent molecular weight of about 300 kDa as determined by SDS-PAGE analysis; p200 of HV-2_{ROD} having an apparent molecular weight of about 200 kDa as determined by SDS-PAGE analysis; p90/80 of HIV-2_{ROD} having an apparent molecular weight of about 90 kDa to about 80 kDa as determined by SDS-PAGE analysis; and gp300 of SIV_{MAC} having an apparent molecular weight of about 300 kDa as determined by SDS-PAGE analysis.

The immune complex as described above, wherein the antibody, protein, and both the antibody and protein, (hereby denoted in this Example as ‘the probe’) are labeled with fluorescent labels, chemiluminescent labels, or chromophore labels, changing the appearance of the label in presence of said envelope proteins of HIV-2 in humane fluids.

The condom utilized in the invention was conveniently manufactured by coating a former with latex r and allowing the coating material to cure if required before the condom is stripped from the former. The coating was a dip molding process in which a shaped former is dipped into a liquid bath e.g. of latex, and withdrawn carrying with it a thin layer of the coating material. The manufacture by dip molding was approached by multiple dips into the same liquid baths thus used to control the thickness of the molded condom. The former was provided with annular grooves for forming the circumferential grooves of the condom and due to natural flow effects during the dipping process the liquid coating at the bottoms of the grooves were slightly thicker than on the other surface portions of the former.

The probe was incorporated onto said organic polymer at the tip of the condom at the exterior side (e.g, the vaginal interface of the article).

### Example 2

Syphilis Detecting Condom

According to U.S. Pat. No. 4,081,334, which entire disclosure of this application is incorporated herein by reference, one milliliter of Ogata antigen, which consists of 0.01% cardiolipin, 0.04% lecithin, 0.20% cholesterin in ethanol and which is used in the Wasserman test for syphilis, was mixed with 6 milliliters of an acetone solution containing 250 milligrams of acetyl cellulose. The mixed solution was caste on a glass plate (18×10 cm²), and dried at room temperature under reduced pressure, after which the cast membrane is peeled off the glass plate. Thus, an immobilized antigen membrane is obtained. This antigen probe was incorporated on a tip of a condom as described above.

### Example 3

Hepatitis B Detecting Surgical Glove

According to EP993470A2, which entire disclosure of this application is incorporated herein by reference, antibodies and other binding molecules specific for S-region of hepatitis B viral antigens (HBV), peptides comprising epitopes recognized by such molecules, were incorporated on surgical gloves as described above in Experiment #1.

### Example 4

Herpes Virus Detecting Condom

According to JP56031646, which entire disclosure of this application is incorporated herein by reference, herpes virus was cultivating through the use of cell such as VERO cell or hatched egg. This antigen was fed to the solution of polystyrene latex having the specific gravity of 1.1 or more and was floated in the phosphate buffer salt solution to obtain the sensitized latex. The herpes virus-antigen containing polymeric solution (hereby denoted in this Example as ‘the probe’) was suspended in a preservation liquid. The sensitized latex liquid was diluted with the efficient amount of dilution liquid. The probe was incorporate at the tip of a condom as described above. Agglutination at the probe contiguous location was indicative for herpes virus presence in the solution.

### Example 4

Neisseria gonorrhoeae Detecting Condom

Gonorrhea is one of the most commonly reported bacterial infections. The causative agent, Neisseria gonorrhoeae, is typically identified by culturing on selective agar medium, gram-staining, and cytochrome oxidase and carbohydrate utilization testing. Known tests are usually sufficient to discriminate N. gonorrhoeae from other Neisseria species. Commercial serological assays, including coagglutination and fluorescent antibody staining, have also been described for the identification of N. gonorrhoea. Knapp, supra.

According to U.S. Pat. No. 5,525,717, which entire disclosure of this application is incorporated herein by reference, a polynucleotide probe specific for N. gonorrhoeae was used. By using several different probes, greater sensitivity of the assay was achieved. This is due to the fact that each probe would hybridize to a different area of the N. gonorrhoeae nucleic acid, so that was each probe was labeled, the analyte had bared multiple labels. The probe was incorporated on a condom as described above.

### Example 5

Male Sperm Detecting Condom

According to U.S. Pat. No. 5,605,803, which entire disclosure of this application is incorporated herein by reference, SP-10 is a sperm-specific antigen identified as an acrosomal constituent present throughout spermiogenesis. The SP-10 gene has been localized to human chromosome 11, and to the junction of bands q23-24. The amino acid sequence has set fort, see Wright et al, Biology of Reproduction, 42:693-201 (1990) and by others. A monoclonal antibody specific for this tissue-specific antigen has been previously developed, identified as MHS-10 and denoted hereby in this Experiment as ‘the probe’. This probe is expressed by a hybridoma cell line deposited at the American Type Culture Collection, 12301 Parklawn Drive, Rockville, Md., under accession number ATCC HB 10039. The probe was incorporate in a condom as descried above.

1. A prophylactic article, useful for both protection and diagnosis of biological, chemical or physical specific conditions in mammals body comprising at least one probe, having means for diagnosis of said conditions

2. A prophylactic article, according to claim 1 wherein the prophylactic article is a male condom or a female condom, useful for protection from sexually transmitted diseases, STDs, and from unintended pregnancy; comprising at least one probe that enables rapid arid sensitive diagnosis of said STDs.

3. A prophylactic article, comprising condom according to claim 2, wherein said probe comprising at least one biological substance of the group of immune complexes, proteins, antibodies, antigens and nucleotides; and means for detecting whether the specific binding substance is bound to at least one of the group: an STD, STD's expression and to an STD vector, microorganism particle or antigen.

4. A prophylactic article, comprising condom according to claim 2, whereas said probe have means of diagnosis of at least one of the known STD comprising by not limited to Acquired Immune Deficiency Syndrome, AIDS; Bacterial Vaginosis, Gardnerella vaginitis; chlamydia; Pediculosis pubis, crab lice; Neisseria gonorrhea; Hepatitis B; herpes virus; Human Papillomavirus, HPV; Molluscum Contagiosum; Scabies; Syphilis; Trichomoniasis, Trich).

5. A prophylactic article, comprising condom according to claim 2, whereas said probe having means for sensitive screening for the presence of male sperm at the vaginal side of said article, and wherein said article is comprising a specific binding substance and means for detecting whether the specific binding molecule is bound to male sperm.

6. A prophylactic article, condom as defined in claim 5, whereas said probe is a sperm-specific antigen.

7. A prophylactic article, according to claim 1, wherein said article is selected from the group of surgical gloves, non-disposable gloves, wound dressings, artificial skin, implements, cloths and coatings.

8. A prophylactic article, comprising surgical glove according to claim 7, whereas said probe have means for protection and for the diagnosing of various conditions related to the presence of at least one from the group of contaminated diseases, disease vectors, hazardous chemicals, and physical conditions.

9. A method for the use prophylactic articles as defined in claim 1, useful for both protection and for biological, chemical or physical specific conditions as described in all claims above, wherein comprising
i. locating an assay probe on said prophylactic article for the detection of condition to be tested in one or more sites on said article and fitting by means of adjustment said article on an object organ;
ii. bringing at least one surface of said organ to be tested and at least one surface of said article into contact for an efficient period of time;
iii. measuring biological, chemical or physical specific conditions diagnosing by means of probe detecting, screening, or analyzing of said condition.

10. A method for the use said prophylactic article, as defined in claim 1, comprising male condom or female condom, useful for both protection and for diagnosing at least one conditions of STDs and presence of male sperm in the vaginal side of said article; wherein comprising
i. locating an assay probe on said condom for the detection of condition to be tested in one or more sites on said article and fitting by means of adjustment said article on an object organ;
ii. bringing at least one surface of said organ to be tested and at least one surface of said article into contact for an efficient period of time;
iii. measuring said condition by means of probe detecting, screening, or analyzing of said condition.

11. A method for the use prophylactic article, comprising surgical gloves, and non-disposable gloves as defined in claim 7, useful for protection and for the diagnosing of various conditions related to the presence of at least one from the group of contaminated diseases, disease vectors, hazardous chemicals, and physical conditions, wherein comprising
i. locating an assay probe on said article for the detection of condition to be tested in one or more sites on said article and fitting by means of adjustment said article on an object organ;
ii. bringing at least one surface of said organ to be tested and at least one surface of said article into contact for an efficient period of time;
iii. measuring said condition by means of probe detecting, screening, or analyzing of said condition.

12. A method for the production of prophylactic article as defined in claim 1, comprising
i. preparing the prophylactic article by an organic polymer selected from the group consisting of polyvinyl chloride, latex, polyurethane, polyacrylate, polyester, polyethylene terephthalate, polymethacrylate, silicone rubber, silicon elastomers, polystyrene, polycarbonate and polysulfones;
ii. absorbing, immobilizing, entrapping or inserting at least one probe onto the organic polymer at the proper location;
iii. and optionally, incorporating suitable diagnosis reference onto the organic polymer at predetermined location.
